(19) United States
(12) Patent Application Publication
(10) Pub. No.: US 2007/0208310 A1
(43) Pub. Date: Sep. 6, 2007

(54) THUMB RING INSERT FOR A SYRINGE

(76) Inventor: Eva Stadick, Maple Valley, WA (US)

Correspondence Address: DAVID L. TINGEY;LAW OFFICE OF DAVID L. TINGEY, 15 SOUTH GRADY WAY, SUITE 336, RENTON, WA, 98057, US

(21) Appl. No.: 11/308,023

(22) Filed: Mar. 3, 2006

(51) Int. Cl. A61M 5/00

(52) U.S. Cl. 604/187

(57) ABSTRACT

A thumb ring insert that reduces the effective size of a syringe thumb ring thumb passage. The insert comprises a thumb aperture adapted to receive a user's thumb in a comfortable fit with the aperture eccentric from the thumb ring passage, located close to a thumb ring bottom where an end of a syringe plunger connects to the thumb ring. Thus, range of movement between aspiration and expression of the syringe is reduced as free float of the thumb in the thumb ring is effectively eliminated. With the insert aperture close to the thumb ring bottom, the expanse between the user's thumb and fingers is also reduced when aspirating. The insert further comprises a moderately pliable circumference groove about the insert that matches the syringe thumb ring receivable therein by snapping the insert into place in the thumb ring passage with groove rims being resiliently distorted in allowing the thumb ring to pass into the groove.

### BACKGROUND

1. Field of the Invention

This invention relates to syringes, and more particularly to an insert for a thumb ring of a syringe and typically for a dental aspirating syringe.

2. Prior Art

Syringes are commonplace in the medical and dental communities, comprising a barrel enclosing a cylindrical cavity through which a plunger moves and including a finger grip on a plunger end, a thumb ring, a needle on a barrel needle end, with a plunger end connected to the thumb ring. In use, medicaments are expressed through the needle by depressing the thumb ring, which in turn presses the plunger through the cavity. By pulling the thumb ring and plunger away from the barrel needle end, blood is aspirated into the cavity if the needle is within a vascular lumen. The plunger is depressed or withdrawn through the cavity by action of a user's thumb in the thumb ring as the user with his fingers on the finger grip squeezes his finger and thumb together to express medicament or separates the fingers and thumb to aspirate fluid or blood.

Close control, or stabilization, is mandatory to properly aspirate and express local anesthetic or other medicament to lower risk of bruising and achieve other desired affects. The syringe is balanced on the index and middle fingers at one point of contact for stabilization and on the thumb for the second point of stabilization causing a base on the hand for the syringe to sit. Often users have a tendency to move the needle of the syringe away from the desired depository site if the user does not have proper stabilization of the syringe. Loss of stabilization can be exacerbated if the user's thumb is considerably smaller than the syringe thumb ring allowing lack of contact with the thumb ring, or free floating of the thumb while moving from expressing to aspirating and back. This free floating eliminates one of two main points of stabilization. A user with a small thumb will experience difficulty using a syringe with a thumb ring sized to fit a larger thumb. The thumb will consequently lose close control of the plunger as the thumb attempts to move between pushing on a thumb ring bottom during expressing medicament and pulling against a thumb ring top during aspiration as the thumb free floats between the two positions. Although the issue could be resolved by using syringe with a smaller thumb ring, often a dental office will have one size of syringe and thumb ring for use by all personnel. Also it is inconvenient during procedure setup to require different syringes for different users.

It is therefore an object of the invention to provide a thumb ring insert that fills the thumb passage through the thumb ring except for a new, smaller thumb aperture in the insert sized to fit users with small thumbs. It is further object to provide thumb ring inserts with respective selectively-sized thumb apertures more precisely fitting a plurality of users with different sized thumbs. It is a further object that the thumb ring aperture be located in the insert closer to the thumb ring bottom, eccentric from the thumb ring passage, thereby reducing the required reach of the thumb to positions corresponding to full plunger depression on fluid expression and full plunger extension, or withdrawal, on aspiration. It is a further object that the insert be adapted for facile insertion and removal from the thumb ring.

### SUMMARY

These objects are achieved in a thumb ring insert that reduces the effective size of a syringe thumb ring thumb passage. The insert comprises a thumb aperture adapted to receive a user's thumb in a comfortable fit only slightly larger than the user's thumb with the aperture eccentric from the thumb ring passage, located close to a thumb ring bottom where a syringe plunger end connects. Thus, the reach, or range of movement, between limiting positions of the plunger in the syringe is reduced as free float of the thumb in the thumb ring is effectively eliminated. With the insert aperture close to the thumb ring bottom, the expanse between the user's thumb and fingers during aspiration is also reduced. That is, the thumb range of motion begins with the thumb as close to the fingers as practical, effectively the position of the thumb in the thumb ring without the insert installed. The reach is then the extent of movement of the plunger in the syringe. In practice, with medicament in the syringe the plunger is mostly withdrawn from the syringe so the thumb is largely extended. Proper aspiration requires only 1 or 2 mm of plunger movement so the thumb is only required to pull out against the thumb ring a small distance for aspiration before pushing the plunger at least partially through the syringe cavity.

The thumb ring insert provides a comfortable fit and convenient aspiration motion, which encourages aspiration prior to deposition of the medicament. Aspiration prior to deposition avoids inadvertent intravascular administration of local anesthetics, which is the standard of care.

The convenience of use of the insert continues with a moderately pliable circumference groove about the insert that matches the syringe thumb ring receivable therein by snapping the insert into place in the thumb ring passage by urging the annular thumb ring into the groove with groove rims being temporarily slightly distorted in allowing the thumb ring to pass into the groove. The insert is similarly removed from the thumb ring with the groove rims, strong enough to hold the insert in the thumb ring passage generally, to distort under application of outside force in allowing the thumb ring to pass out of the groove.

### BRIEF DESCRIPTION OF THE DRAWINGS

FIG. 1 is a front view of a typical syringe with a thumb ring.

FIG. 2 are front and side views of the insert of the present invention.

FIG. 3 is a front view of the insert in the syringe thumb ring shown in full aspiration position with full expression position shown in phantom lines.

FIG. 4 is an artistic view of the syringe with insert held in operational position by a user's hand.

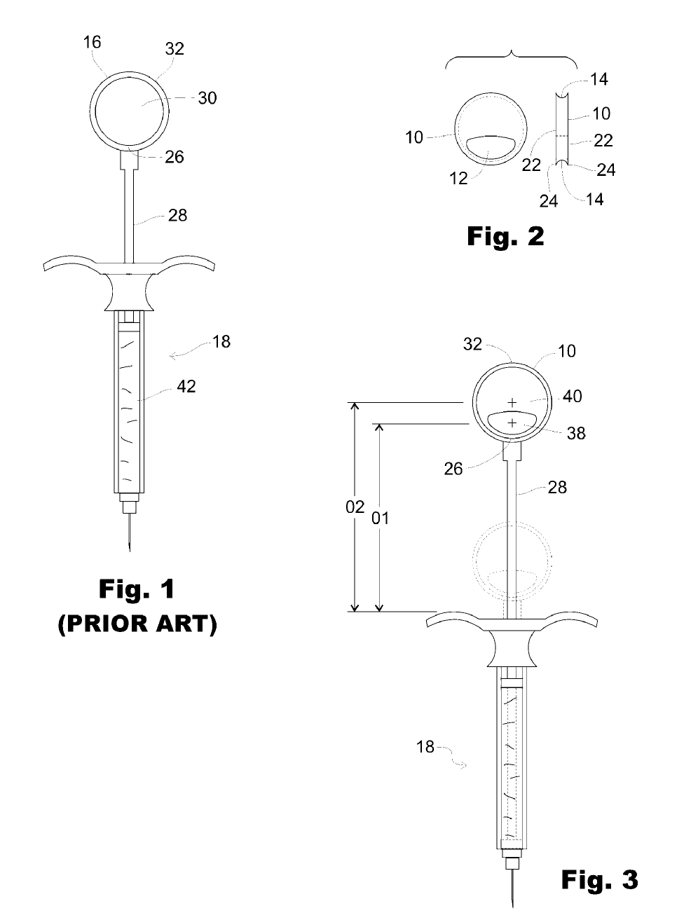

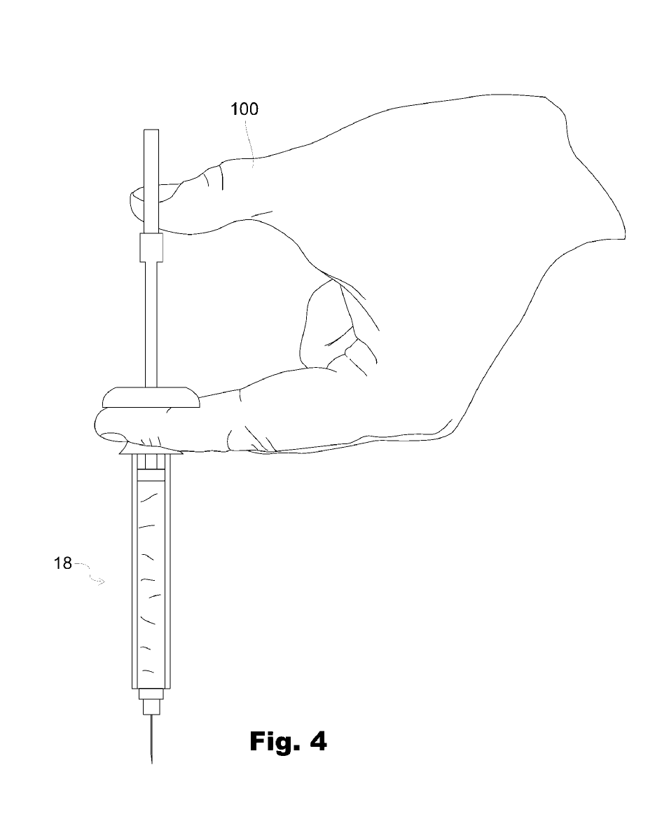

### DETAILED DESCRIPTION OF THE PREFERRED EMBODIMENT

The syringe insert 10 of the present invention has a through thumb hole 12 for receiving a user's thumb 100 and with a circumferential groove 14 sized to match an annular thumb ring 16 of a syringe 18 with the thumb ring 16 removably fitting in an insert circumferential groove 20. Typically, the insert 10 is disk-shaped comprising parallel sides 22 with the insert groove 14 between them at their circumference. The circumferential groove 14 comprises resilient sides 24 such that the thumb ring 16 distorts at least a portion of the groove sides 24 as the thumb ring 16 slides over the groove sides 24 into the groove 14, the groove sides 24 resiliently returning to nondistorted form when the thumb ring 16 is installed in the groove 14. The insert 10 is thus secured to the thumb ring 16.

The insert thumb hole 12 is located near a thumb ring bottom 26 to which a syringe plunger 28 attaches, thereby functionally reducing the size of thumb ring opening 30 to the size of the insert thumb hole 12 and reducing or eliminating the free float of a user's thumb 100 between the thumb ring bottom 26 and its top 32 (opposite the ring bottom) therein reducing range of movement of the thumb 100 between aspiration and expression of fluid into and out of syringe 34. For these purposes, free float of the thumb 100 means noncontact of the thumb 100 with the thumb ring bottom 26 (typical for expression of fluid from the syringe) or the ring top 32, (typical for aspiration of fluid into the syringe 34), the range of thumb movement thus being between contact with the ring top 32 and bottom 26.

The distance D1 between syringe finger grip 36 and an insert hole center 38 is less than the distance D2 between the finger grip 36 and a thumb ring opening center 40, thereby reducing a range of thumb movement between aspiration and expressing syringe medicament. For these purposes, expressing or expression of fluid or medicament from a syringe means expulsion of the fluid or medicament from the syringe cavity 42 by downward movement of the syringe plunger.

In use, the insert 10 is selectively chosen from an assembly of similar inserts having differing hole sizes such that the insert hole matches a user's thumb therein precluding free float of the thumb between aspiration and medicament expression.

What is claimed is:

1. A syringe including a barrel enclosing a cylindrical cavity through which a plunger moves, a needle on a barrel needle end, a finger grip on a barrel plunger end, an annular thumb ring with an opening through the thumb ring, a plunger with an end connected to the thumb ring at a thumb ring bottom, the improvement comprising a removable insert within the thumb ring opening with the thumb ring receivable into an insert groove about the insert circumference that matches the thumb ring, the insert having a thumb hole adapted to receive a user's thumb, the thumb hole located near the thumb ring bottom functionally reducing the size of the thumb ring opening to the size of the insert thumb hole.

2. A syringe including a barrel enclosing a cylindrical cavity through which a plunger moves, a needle on a barrel needle end, a finger grip on a barrel plunger end, an annular thumb ring with an opening through the thumb ring, a plunger with an end connected to the thumb ring at a thumb ring bottom and the plunger, the improvement comprising an insert removably within the thumb ring opening with the thumb ring receivable into an insert groove about the insert circumference that matches the thumb ring, the insert having a thumb hole adapted to receive a user's thumb, the thumb hole located near the thumb ring bottom functionally reducing the size of the thumb ring opening to the size of the insert thumb hole.

3. The syringe of claim 2 wherein the insert is disk-shaped comprising parallel sides with the insert groove between them at their circumference.

4. The syringe of claim 2 wherein the insert comprises resilient groove sides such that the thumb ring distorts at least a portion of the groove sides as the thumb ring slides over the sides into the groove, the groove sides resiliently returning to nondistorted form when the thumb ring is installed in the groove therein securing the insert to the thumb ring.

5. The syringe of claim 2 wherein the distance between the finger grip and an insert hole center is less than the distance between the finger grip and a thumb ring opening center, thereby reducing a range of thumb movement between aspiration and expressing syringe medicament.

6. The syringe of claim 2 wherein the insert is selectively chosen such that the insert hole matches a user's thumb therein precluding free float of the thumb between aspiration and medicament expression.

7. A syringe insert with a through hole for receiving a user's thumb and with a circumferential groove sized to match an annular thumb ring of a syringe with the thumb ring removably fitting in the circumferential groove, the circumferential groove comprising resilient sides such that the thumb ring distorts at least a portion of the groove sides as the thumb ring slides over the sides into the groove, the groove sides resiliently returning to nondistorted form when the thumb ring is installed in the groove therein securing the insert to the thumb ring, wherein the thumb hole is located near a thumb ring bottom to which a syringe plunger attaches, thereby functionally reducing the size of the thumb ring opening to the size of the insert thumb hole and reducing or eliminating the free float of a user's thumb between the thumb ring bottom and its top (opposite the bottom) therein reducing range of movement of the thumb between aspiration and expression of fluid into and out of the syringe, wherein the distance between the finger grip and an insert hole center is less than the distance between the finger grip and a thumb ring opening center, thereby reducing a range of thumb movement between aspiration and expressing syringe medicament, and wherein the insert is selectively chosen such that the insert hole matches a user's thumb therein precluding free float of the thumb between aspiration and medicament expression.

8. The syringe of claim 7 wherein the insert is disk-shaped comprising parallel sides with the insert groove between them at their circumference.
